(19) United States
(12) Patent Application Publication
(10) Pub. No.: US 2011/0295021 A1
(43) Pub. Date: Dec. 1, 2011

(54) METHOD OF CRYSTALLIZING CARNOSOL

(76) Inventor: Christof Wehrli, Witterswil (CH)

(21) Appl. No.: 13/120,603

(22) PCT Filed: Sep. 15, 2009
(86) PCT No.: PCT/EP2009/061929

§ 371 Date: Jun. 13, 2011

(30) Foreign Application Priority Data
Sep. 30, 2008 (EP) 08017178.8

(51) Int. Cl. C07D 493/08

(52) U.S. Cl. 549/278

(57) ABSTRACT

Carnosol can be crystallized from a plant extract by contacting the extract with acetic acid, and collecting the crystals so formed.

### FIELD OF THE INVENTION

This invention relates to a method of crystallizing carnosol from a solution.

### BACKGROUND OF THE INVENTION

Carnosol is a naturally occurring compound in a number of plant species such as sage, Greek sage, and most notably, rosemary. Recently, there have been a number of investigations into its biological activitiy. See, for example WO 07/131767 which describes its use against prostate carcinoma, and WO 08/061755 which describes uses to improve disorders associated with impaired neurotransmission.

Crude commercially available rosemary extract generally contain about 20% -60% of abietanes (where carnosol is only a part), and of about 1-80% of many different compounds of mainly unknown structure. In order to isolate the carnosol, multi-step processes are often employed, which add expense to the final product.

Further, in many commonly used organic solvents allowed for food processing (food grade solvents), such as acetone, it is not possible to crystallize carnosol from rosemary extracts, or only a small portion can be crystallized (e.g. from ethanol), making the direct crystallization of carnosol from rosemary extracts economically not attractive.

There is a need for carnosol of higher purity, and for a simple and efficient method of obtaining it.

### DETAILED DESCRIPTION OF THE INVENTION

This invention relates to a novel method of crystallizing carnosol from a solution comprising at least 5% carnosol comprising: contacting the solution with acetic acid to form carnosol crystals. If desired, the crystals may then be separated from the extract using conventional methods such as filtering and drying.

The solution is preferably a plant extract, but it may be a waste stream from the production of plant extract. As long as it contains at least 5% carnosol, it is not critical to the practice of this invention. If the carnosol concentration falls below 5%, it is unlikely that the process will be enconomical enoungh to be a commercially viable process, even though some success at crystallization may be observed under certain conditions.

The plant extract may be from any species of plant or mixture of plant species, as long as it contains carnosol. In preferred embodiments, plants known to contain relatively high amounts of carnosol such as rosemary or sage are used as the source of the carnosol. Many are commercially available from a variety of producers.

The type of plant extract, i.e. water, organic solvent, supercritical fluid (such as supercritical carbon dioxide, etc.) or a mixture thereof is not particularly critical for the practice of this invention, as long as the extract contains some amount of carnosol. Further, the starting material for this invention may be a plant extract which is commercially available, and /or a plant extract which has already been subjected to further processing steps. Further, the plant extract may be “crude” and contain a relatively low amount (i.e. 5-20%) of carnosol and still be used as a source of carnosol crystals according to this invention.

Prior to the crystallization step, if desired, carnosic acid which may be present in the extract may be converted to carnosol. Details on this optional step are taught in co-pending patent applications EP 08007339.8 and 08161724.3

Any commonly used acetic acid can be used; food grade acetic acid of >95% content is preferred. The temperature of the crystallization is not critical as long as it is above the freezing point of the mixture; ambient is preferred. The ratio of extract:acetic acid is not critical, however a ratio of 1:2 to 1:10 is preferred and more preferred is a ratio of 1:3 to 1:5.

The following non-limiting examples are presented to further illustrate the invention.

### EXAMPLE 1

### Comparative Example

10 g of Commercially available rosemary extract A (43% carnosic acid, 18% carnosol) was stirred in 40 ml acetone for 1 d at ambient. No carnosol crystallised.

### EXAMPLE 2

10 g rosemary extract of Commercailly available rosemary extract A (43% carnosic acid, 18% carnosol) was stirred in 40 ml acetic acid for 1 d at ambient. The slurry was filtered and the crystals washed with 8 ml of acetic acid. The crystals where dried in the vacuum at 70° C. We obtained 1.96 g carnosol of 78% purity (yield=83%)

### EXAMPLE 3

Examples 3a-3b where conducted similar to example 2

| | Rosemary | Carnosol | | Carnosol crystallised | | |
|---|---|---|---|---|---|---|
| No | extract | in Extract | Solvent | [g] | purity | Yield |
| 1 | A | 18.3% | Acetone | No crystallization | | — |
| 3a | A | 18.3% | Ethanol | 1.15 g | 85.6% | 43% |
| 2 | A | 18.3% | Acetic acid | 3.05 g | 77.5% | 83% |
| 3b | B | 15.9% | Acetic acid | 0.52 g | 79.6% | 63% |
| 3c | C | 8.4% | Acetic acid | 0.90 g | 55.2% | 59% |
| 3d | D | 6.5% | Acetic acid | 0.17 g | 58.1% | 25% |

Purity and contents determined by HPLC analysis as [weight %]

1. A method of obtaining carnosol crystals comprising contacting a solution comprising at least 5% carnosol with acetic acid.

2. A method according to claim 1 wherein the solution comprises a plant extract.

3. A method according to claim 1, wherein the plant extract is a rosemary extract.

4. A method according to claim 1, further comprising separating the crystals so formed from the solution.

5. A method according to claim 2, wherein prior to contacting with acetic acid, at least some of carnosic acid contained in the plant extract is converted to carnosol.

6. The use of carnosol crystals produced by claim 1, as a dietary supplement, pharmaceutical ingredient, a cosmetic ingredient, as antioxidant or a food ingredient.
